(19) United States
(12) Patent Application Publication
(10) Pub. No.: US 2025/0336528 A1
(43) Pub. Date: Oct. 30, 2025

(54) FALL PREVENTION CONSOLIDATED VIEW AND ALERTING

(71) Applicant: Hill-Rom Services, Inc., Batesville, IN (US)

(72) Inventors: Patrice Etchison, Cary, NC (US); Sarah E. Blanck, Highland Park, IL (US); Chad G. Malone, Vernon Hills, IL (US); Megan R. Melvin, Vernon Hills, IL (US); Traci L. Reed, Nashville, TN (US)

(21) Appl. No.: 19/189,545

(22) Filed: Apr. 25, 2025

Related U.S. Application Data
(60) Provisional application No. 63/639,094, filed on Apr. 26, 2024.

(51) Int. Cl. G16H 50/20; G16H 10/60; G16H 40/20; G16H 50/30

(52) U.S. Cl. CPC G16H 50/20; G16H 10/60; G16H 40/20; G16H 50/30

(57) ABSTRACT

A system for improving falls risk protocol compliance receives health data from a plurality of data sources. The system filters the health data for data relevant to a falls risk for a patient. The system identifies a falls risk protocol for the patient. The system displays the data relevant to the falls risk for the patient in a consolidated view, and generates an alert for compliance with the falls risk protocol for the patient.

### CROSS-REFERENCE TO RELATED APPLICATION

This application claims the benefit of and priority to U.S. Provisional Patent Application No. 63/639,094, filed Apr. 26, 2024, the entire disclosure of which is incorporated by reference herein in its entirety.

### BACKGROUND

Patients in healthcare facilities, such as hospitals, clinics, nursing homes and the like are often in compromised medical conditions. Injuries sustained by patients due to falls in healthcare facilities result in significant healthcare costs. In an effort to prevent such injuries, various protocols are implemented to mitigate the risks. For example, patients who are at risk of falling when moving unassisted may be identified as fall risks, and certain protocols may be implemented to reduce the opportunity for the patients to move about unassisted.

The current state of fall prevention typically includes completing a falls risk assessment within an electronic health record (EHR) flowsheet that varies from healthcare facility to healthcare facility. Typically, there is no indicator to identify when a new falls risk assessment should be completed based on changing patient conditions. Following a falls risk assessment, clinicians follow fall risk-associated protocols that are individualized per healthcare facility and are not automated to prompt action or drive accountability for task completion. Accordingly, there is a need for an integrated solution that provides a view to access, triage, and document fall prevention data points with indicators to drive safety protocol adherence.

Further, accurate assessment of fall risk is relevant for efficient allocation of hospital resources to prevent patient falls. However, accurate assessment of fall risk is challenging in view of a large number of risk factors. Further, such risk factors are continuously changing based on new research updating the body of knowledge regarding fall risk factors and how to weigh each one. Accordingly, it would be desirable to have an integrated solution that proactively addresses fall risks and drives fall protocol adherence to mitigate risk.

### SUMMARY

In general terms, the present disclosure relates to fall prevention. In one possible configuration, data relevant to falls risk for a patient is displayed in a consolidated view, and an alert is generated for compliance with a falls risk protocol for the patient. Various aspects are described in this disclosure, which include, but are not limited to, the following aspects.

One aspect relates to a system for improving falls risk protocol compliance, the system comprising: at least one processing device; and at least one computer readable data storage device storing software instructions that, when executed by the at least one processing device, cause the at least one processing device to: receive health data from a plurality of data sources; identify data relevant to a falls risk for a patient; identify a falls risk protocol for the patient; display the data relevant to the falls risk for the patient in a consolidated view; and generate an alert for compliance with the falls risk protocol for the patient.

Another aspect relates to a method for improving falls risk protocol compliance, the method comprising: receiving health data from a plurality of data sources; identifying data relevant to a falls risk for a patient; identifying a falls risk protocol for the patient; displaying the data relevant to the falls risk for the patient in a consolidated view; and generating an alert for compliance with the falls risk protocol for the patient.

Another aspect relates to a non-transitory computer readable storage media including computer readable instructions which, when read and executed by a computing device, cause the computing device to: receive health data from a plurality of data sources; identify data relevant to a falls risk for a patient; identify a falls risk protocol for the patient; display the data relevant to the falls risk for the patient in a consolidated view; and generate an alert for compliance with the falls risk protocol for the patient.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a healthcare facility that includes a risk consolidating system for assessing risk for a patient in a patient environment.

FIG. 2 illustrates an example of an exterior of the patient environment of FIG. 1.

FIG. 3 schematically illustrates an example of the risk consolidating system of FIG. 1.

FIG. 4 schematically illustrates an example of a method of improving falls risk protocol compliance that can be performed by the risk consolidating system of FIG. 1.

FIG. 5 illustrates an example of a consolidated view that can be generated by a data visualization module installed on the risk consolidating system of FIG. 1.

FIG. 6 illustrates another example of a consolidated view that can be generated by the data visualization module installed on the risk consolidating system of FIG. 1.

FIG. 7 illustrates an example of a bed data tab that is displayed when selected from the consolidated view of FIG. 6.

FIG. 8 illustrates an example of fall risk care plan that can be displayed when the documentation tab is selected in the consolidated view of FIG. 6.

FIG. 9 illustrates an example of a triage view that can be generated by the data visualization module installed on the risk consolidating system of FIG. 1.

FIG. 10 illustrates another example of a triage view that can be generated by the data visualization module installed on the risk consolidating system of FIG. 1.

FIG. 11 illustrates an example of an alert that can be generated by a risk protocol alert module installed on the risk consolidating system of FIG. 1.

FIG. 12 illustrates another example of an alert that can be generated by the risk protocol alert module installed on the risk consolidating system of FIG. 1.

FIG. 13 illustrates another example of an alert that can be generated by the risk protocol alert module installed on the risk consolidating system of FIG. 1.

FIG. 14 illustrates another example of an alert that can be generated by the risk protocol alert module installed on the risk consolidating system of FIG. 1.

FIG. 15 illustrates another example of an alert that can be generated by the risk protocol alert module installed on the risk consolidating system of FIG. 1.

FIG. 16 illustrates another example of an alert that can be generated by the risk protocol alert module installed on the risk consolidating system of FIG. 1.

FIG. 17 illustrates another example of an alert that can be generated by the risk protocol alert module installed on the risk consolidating system of FIG. 1.

FIG. 18 illustrates another example of an alert that can be generated by the risk protocol alert module installed on the risk consolidating system of FIG. 1.

FIG. 19 illustrates another example of an alert that can be generated by the risk protocol alert module installed on the risk consolidating system of FIG. 1.

FIG. 20 illustrates another example of an alert that can be generated by the risk protocol alert module installed on the risk consolidating system of FIG. 1.

FIG. 21 illustrates another example of an alert that can be generated by the risk protocol alert module installed on the risk consolidating system of FIG. 1.

### DETAILED DESCRIPTION

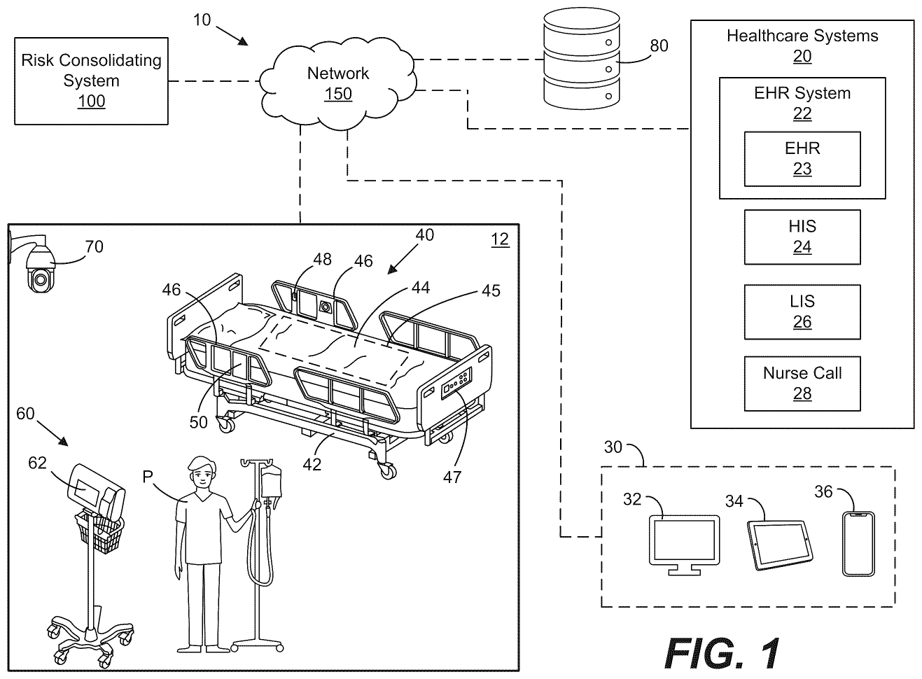

FIG. 1 illustrates an example of a healthcare facility 10 that includes a risk consolidating system 100 for improving falls risk protocol compliance for a patient P in a patient environment 12. As will be described in more detail, the risk consolidating system 100 displays data relevant to falls risk for a patient in a consolidated view, and generates alerts for compliance with a falls risk protocol for the patient. While the following description refers to assessing falls risk, the risk consolidating system 100 may also be configured to assess other types of risk.

Examples of the healthcare facility 10 can include hospitals, long-term care facilities, nursing homes, surgery centers, and the like. Examples of the patient environment 12 can include a room or area within the healthcare facility 10 such as a patient room.

The patient environment 12 includes medical devices and other equipment such as a patient support apparatus 40, a patient monitoring device 60, and a camera 70. Additional types of medical devices and equipment, or fewer types of medical devices and equipment can be positioned in the patient environment 12 such that the patient support apparatus 40, the patient monitoring device 60, and the camera 70 are shown for illustrative purposes.

The patient support apparatus 40 can be a hospital bed, a stretcher, an operating room table, or similar type of apparatus on which a patient can rest. The patient support apparatus 40 includes a frame 42 supported by wheels or casters, a mattress 44 supported by the frame 42, a number of siderails 46, a headboard, and a footboard, as shown. The siderails 46 can be raised to a deployed position to prevent exit from the patient support apparatus 40 by the patient P, or can be lowered into a stowed position to allow the patient P to exit the patient support apparatus 40.

The patient support apparatus 40 can include one or more sensors 45 positioned under the mattress 44. The one or more sensors can measure one or more physiological parameters such as heart rate, non-invasive blood pressure (NIBP), motion, and weight. Additionally, the one or more sensors 45 can be used detect patient exit, incontinence, deterioration, and other metrics relevant to the health of the patient P. The one or more sensors 45 can further detect whether the patient support apparatus 40 is being occupied by the patient P.

A bed exit alarm 47 can be activated on the patient support apparatus 40. The bed exit alarm 47 is triggered when data captured by the one or more sensors 45 predicts that motion by the patient P indicates that the patient P is attempting to exit the patient support apparatus 40, or that the patient P has already exited the patient support apparatus 40. A sensitivity for triggering the bed exit alarm 47 is adjustable. For example, a high sensitivity can be set for when the patient P has a high falls risk, and a low sensitivity can be set for when the patient P has a low or moderate falls risk. The sensitivity for triggering the bed exit alarm 47 can be based on a threshold for the patient motion indicating a likelihood the patient P is attempting to exit the patient support apparatus 40 where a lower threshold is associated with a higher sensitivity, and a higher threshold is associated with a lower sensitivity. Additional examples are contemplated.

The patient monitoring device 60 can be used to measure and monitor physiological parameters of the patient, and to display representations of the measured physiological parameters on a display 62. In some examples, the display 62 is a touchscreen that operates to receive tactile inputs from a user such as the caregiver C such that the display 62 is both a display device and a user input device. In some examples, the display 62 is a liquid-crystal display (LCD), an organic light-emitting diode (OLED, a plasma panel, a quantum-dot light-emitting diode (QLED), or other type or combination of display screen technology.

The patient monitoring device 60 includes one or more sensor modules that can be used to measure one or more physiological parameters of the patient. For example, the patient monitoring device 60 can include a temperature sensor module for measuring the patient's temperature, a pulse oximetry sensor module for measuring the patient's blood oxygen saturation (SpO2), and a non-invasive blood pressure (NIBP) sensor measurement module for measuring the patient's blood pressure. As used herein, a “module” is a combination of physical structure which resides in the patient monitoring device 60 and peripheral components that attach to and reside outside of the patient monitoring device 60. The patient monitoring device 60 can include additional sensor modules for receiving additional physiological parameter measurements, including, without limitation, heart rate, pulse, and ECG/EKG.

As further shown in FIG. 1, a camera 70 is mounted to a surface of the patient environment 12 such as a wall or ceiling. The camera 70 can be mounted at different locations within the patient environment 12 such that the mounting of the camera 70 as shown in FIG. 1 is provided by way of illustrative example. Alternatively, the healthcare facility 10 can include a plurality of cameras mounted onto multiple surfaces within the patient environment 12.

The camera 70 is configured to pan, tilt, and zoom for adjusting a view of the patient environment 12 as well as views of individual objects within the patient environment 12 such as a patient, the patient support apparatus 40, and a caregiver. The camera 70 can include a gimbal or similar structure actuated by an electric motor to pan the camera 70 left and right, and to tilt the camera 70 up and down. Also, the camera 70 can zoom in and out by adjusting a focal length of a lens whether mechanically (e.g., mechanical zoom) or digitally (e.g., digital zoom).

In some examples, data captured by the camera 70 can be used to trigger the bed exit alarm 47 on the patient support apparatus 40 such as when the data captured by the camera 70 indicates that the patient P is attempting to exit the patient support apparatus 40, or that the patient P has already exited to the patient support apparatus 40. Additionally, the data captured by the camera 70 can be used to monitor the patient P's movements in the patient environment 12, and can be used to detect when the patient P falls inside the patient environment 12.

The risk consolidating system 100 is communicatively coupled via a network 150 to the medical devices and other equipment insides the patient environment 12 such as the patient support apparatus 40, the patient monitoring device 60, and the camera 70. The network 150 can connect and exchange data between the medical devices and equipment inside the patient environment 12, and also connect and exchange data with the risk consolidating system 100.

The network 150 can include any type of wired or wireless connections, or any combinations thereof. Wireless connections can be accomplished using Wi-Fi, ultra-wideband (UWB), Bluetooth, and the like. The network 150 can include an Internet of things (IoT) network that connects and exchanges data over the Internet or other communications networks.

The network 150 also connects and exchanges data between the risk consolidating system 100 and one or more healthcare systems 20 associated with the healthcare facility 10 such as an electronic health record (EHR) system 22, a hospital information system (HIS) 24, a laboratory information system (LIS) 26, and a nurse call system 28.

As described herein, the terms electronic medical records (EMRs) and electronic patient record (EPRs) can be used interchangeably with EHRs. The EHR system 22 collects patient electronically stored health information in a digital format (e.g., EHRs 23). As such, the EHR system 22 maintains a plurality of EHRs 23 for a plurality of patients admitted to the healthcare facility 10. Further, the EHRs 23 can be shared across multiple healthcare facilities through network-connected, enterprise-wide information systems or other information networks and exchanges. The EHRs 23 may include a range of data, including demographics, medical history, medication and allergies, immunization status, laboratory test results, radiology images, vital signs, personal statistics like age and weight, and billing information.

The HIS 24 is a comprehensive, integrated information system that manages operational aspects of the healthcare facility 10 such as medical, administrative, financial, and legal, and corresponding processing of services. The HIS 24 may utilize hospital management software (HMS) such as to coordinate healthcare services in the healthcare facility 10.

The LIS 26 support operations of a laboratory within the healthcare facility 10 such as by providing workflow and data tracking support, data exchange interfaces, and enterprise resource planning tools that manage multiple aspects of laboratory informatics.

The nurse call system 28 connects caregivers in the healthcare facility 10 to the patient P to keep the patient P safe and satisfied while improving the patient P's experience while admitted to the healthcare facility 10. For example, the nurse call system 28 allows the patient P to alert a caregiver of their need for help such as by pressing a nurse call button 48 on the patient support apparatus 40. When the nurse call button 48 is pressed, a signal alerts staff at a nurse's station, and usually, a caregiver such as a nurse or a nurse assistant responds to the call. In some instances, the nurse call button 48 allows the patient P to speak directly to the nurse.

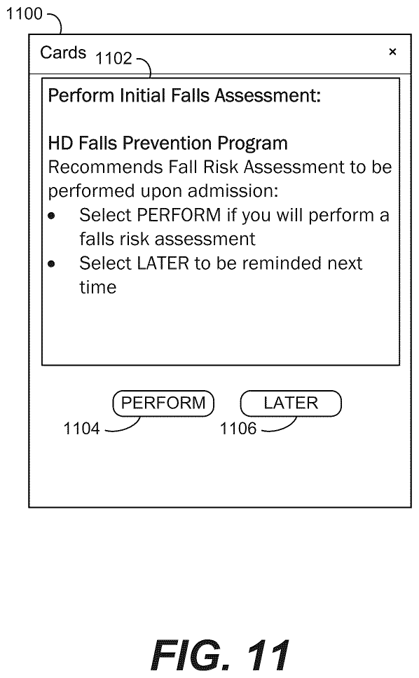

In some examples, the nurse call system 28 communicates alerts that are generated by the risk consolidating system 100 to caregivers via the network 150. Illustrate examples of the alerts that can be generated by the risk consolidating system 100 are shown in FIGS. 11-21.

The risk consolidating system 100 is further communicatively coupled to a protocol database 80 via the network 150. In some examples, the protocol database 80 stores protocols, regulations, and rules that are set and/or customized by the healthcare facility 10 or by a particular unit within the healthcare facility 10. In some examples, the protocols, regulations, and rules stored in the protocol database 80 are mandated based on the geographical location of the healthcare facility 10 such as the state or city where the healthcare facility 10 is located.

The protocols, regulations, and rules stored in the protocol database 80 define actions to mitigate risks for the patients admitted to the healthcare facility. For example, the protocols, regulations, and rules stored in the protocol database 80 can require that a falls risk assessment be performed for each patient upon admission to the healthcare facility 10, that the falls risk assessment be updated at least once per shift or when a patient is transferred to a new unit within the healthcare facility 10, and that a falls risk assessment be updated when a change in medication administered to a patient, a change in the patient's mental status, and the like occurs.

The protocols, regulations, and rules stored in the protocol database 80 can further require that certain actions are implemented based on the falls risk of a patient such as activating the bed exit alarm on the patient support apparatus 40 when the patient has a high or moderate falls risk, altering a display mounted outside the patient environment 12 to display a screen indicating high or moderate falls risk, and other risk mitigating actions.

The risk consolidating system 100 generates alerts to proactively ensure compliance with the protocols, regulations, and rules stored in the protocol database 80. The alerts can require a caregiver accept, decline, or acknowledge a recommended protocol that is retrieved by the risk consolidating system 100 from the protocol database 80. In some examples, the risk consolidating system 100 uses artificial intelligence to generate the alerts based on data acquired from a plurality of sources such as any one of the healthcare system 20 and/or the devices in the patient environment 12, as well as based on the data stored in the protocol database 80. Examples of the alerts generated by the risk consolidating system 100 are shown in FIGS. 11-21.

As further shown in FIG. 1, the risk consolidating system 100 is communicatively connected via the network 150 to a plurality of workstations 30 such as a workstation monitor 32, a portable tablet 34, and a mobile device 36. Additional types of workstations are contemplated such that the workstation monitor 32, the portable tablet 34, and the mobile device 36 are provided for illustrative purposes. The risk consolidating system 100 can display the alerts on the plurality of workstations 30 to proactively ensure compliance with the protocols, regulations, and rules stored in the protocol database 80. The risk consolidating system 100 can also display the alerts on a display 50 on the patient support apparatus 40, on the display 62 of the patient monitoring device 60, and/or on the display 38 mounted on the wall 14 ensure compliance with the protocols, regulations, and rules stored in the protocol database 80.

Additionally, the risk consolidating system 100 can display data relevant to falls risk for a patient in a consolidated view on the plurality of workstations 30. The consolidated view can be displayed within the EHR 23 of the patient P such as on a dedicated tab or window. Accordingly, the data relevant to falls risk that is consolidated by the risk consolidating system 100, as well as the alerts generated by the risk consolidating system 100, can be displayed as an electronic health record (EHR) plug-in of the EHRs 23 stored in the EHR system 22. In some examples, the risk consolidating system 100 also displays the consolidated view that includes the data relevant to falls risk on the display 50 on the patient support apparatus 40, on the display 62 of the patient monitoring device 60, or on any other display of a medical device.

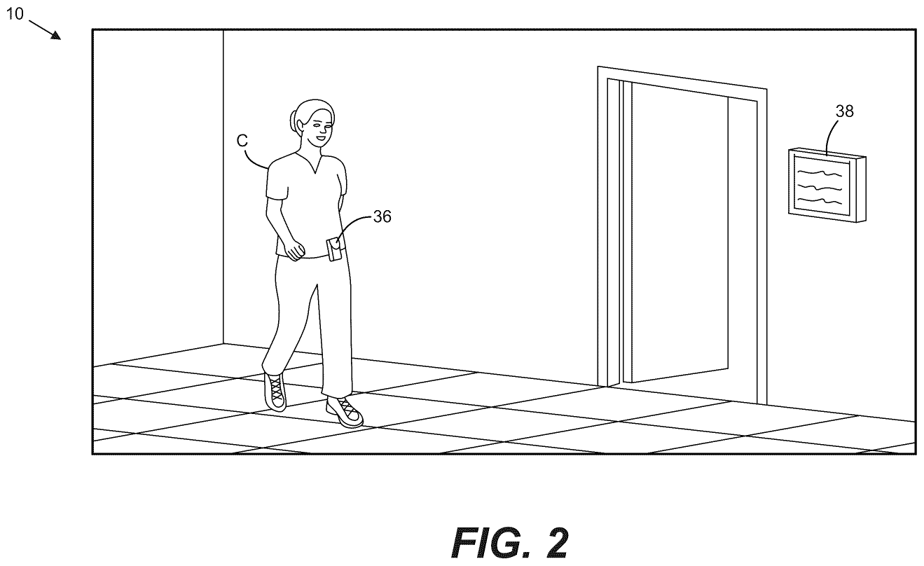

FIG. 2 illustrates an example of an exterior of the patient environment 12. In this example, a display 38 is mounted on a wall 14 proximate to an entrance 16 of the patient environment 12. The healthcare facility 10 can include a display outside each patient environment 12 such that the healthcare facility 10 includes a plurality of displays 38. A caregiver C is shown walking adjacent to the entrance 16 such that the caregiver C can view the display 38 when outside of the patient environment 12. The caregiver C is further shown carrying the mobile device 36. The risk consolidating system 100 can display the alerts on the display 38 to proactively ensure compliance with the protocols, regulations, and rules stored in the protocol database 80. In some examples, the risk consolidating system 100 can also display the consolidated view on the display 38 for displaying data relevant to falls risk for the patient P.

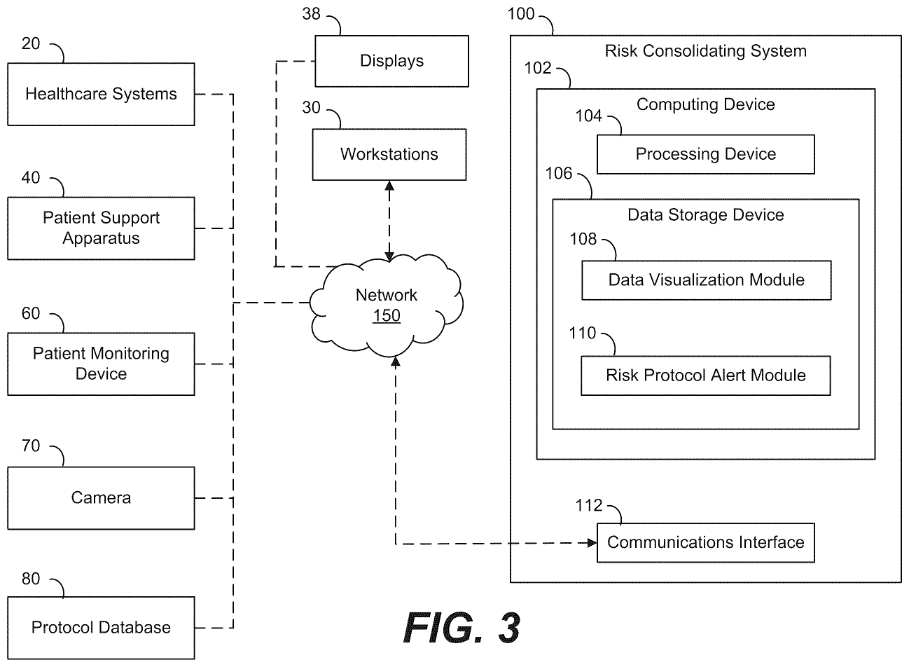

FIG. 3 schematically illustrates an example of the risk consolidating system 100. In FIG. 3, the risk consolidating system 100 is shown as communicatively coupled via the network 150 to the healthcare systems 20, the patient support apparatus 40, the patient monitoring device 60, the camera 70, the protocol database 80, the plurality of workstations 30, and the displays 38 mounted on the walls 14 outside the patient environments 12. The risk consolidating system 100 includes a communications interface 112 that allows the risk consolidating system 100 to connect to the network 150. The communications interface 112 can include wired interfaces and wireless interfaces. For example, the communications interface 112 can wirelessly connect to the network 150 through cellular network communications, Wi-Fi, and other wireless connections. Alternatively, the communications interface 112 can connect to the network 150 using wired connections such as through an Ethernet or Universal Serial Bus (USB) cable.

The risk consolidating system 100 includes a computing device 102 having at least one processing device 104 and at least one computer readable data storage device 106. The at least one processing device 104 is an example of a processing unit such as a central processing unit (CPU). The at least one processing device 104 can include one or more CPUs. In some examples, the at least one processing device 104 includes one or more digital signal processors, field-programmable gate arrays, and/or other types of electronic circuits.

The at least one computer readable data storage device 106 stores data and software instructions for execution by the at least one processing device 104. For example, the at least one computer readable data storage device 106 stores a data visualization module 108 and a risk protocol alert module 110, which are described in more detail below. The at least one computer readable data storage device 106 includes computer-readable media, which includes any media that can be accessed by the at least one processing device 104.

By way of example, computer-readable media include computer readable storage media and computer readable communication media. Computer readable storage media includes volatile and nonvolatile, removable and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the data acquisition device. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

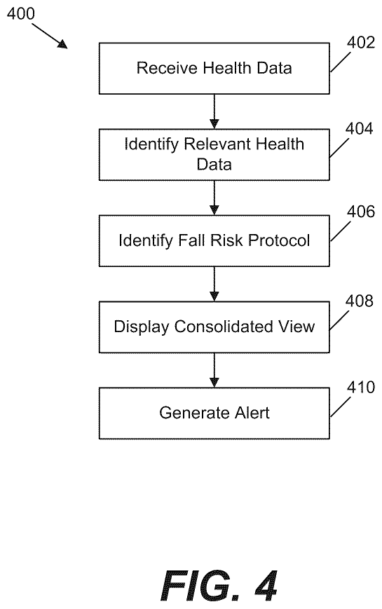

FIG. 4 schematically illustrates an example of a method 400 of improving falls risk protocol compliance. The method 400 can be performed by the risk consolidating system 100.

The method 400 includes an operation 402 of receiving health data from a plurality of data sources. Operation 402 can include receiving health data from one or more of the healthcare systems 20 such as the EHR system 22, the HIS 24, the LIS 26, and the nurse call system 28. Operation 402 can also include receiving the health data from one or more devices inside the patient environment 12 such as the patient support apparatus 40, the patient monitoring device 60, and the camera 70. Additional sources of the health data are contemplated.

The method 400 includes an operation 404 of identifying relevant health data for a risk associated with the patient P. In some examples, operation 404 includes filtering the health data received in operation 402 for data relevant to a falls risk of the patient P. In some examples, the data is filtered to identify health data relevant to assessing a fall risk of the patient. The risk consolidating system 100 may also be configured to assess other types of risk such that operation 404 can include filtering the health data for data relevant to other types of risk.

Operation 404 can include filtering the health data such as patient demographic data, patient status data, labs and vital signs data, and medications for data relevant to assessing a falls risk for a patient such as one or more of patient age, medical diagnoses, mental health status, whether the patient is post-operative, patient weight, blood pressure, gait, and medications that can cause dizziness, sedation, confusion, blurred vision, or orthostatic hypertension.

The method 400 includes an operation 406 of identifying a falls risk protocol for the patient P. Operation 406 can include accessing the protocol database 80 and identifying the falls risk protocol based on at least one of a geographical location of the healthcare facility 10 (e.g., state or city within the United States), a department or unit within the healthcare facility 10, or a patient population which the patient P belongs to (e.g., geriatric, post-surgical, and the like).

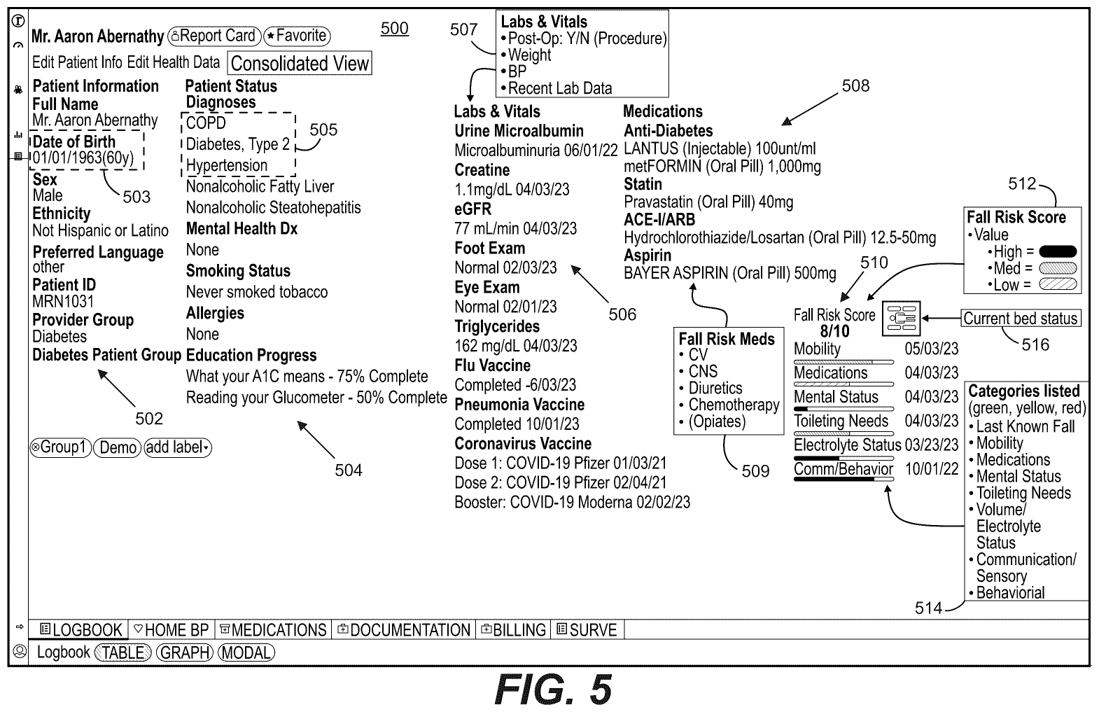

The method 400 includes an operation 408 of displaying the data relevant to the falls risk for the patient in a consolidated view. An example of the consolidated view is shown in FIG. 5, which will be described in more detail further below. Operation 408 can include displaying the consolidated view within the EHR 23 associated with the patient P such that the consolidate view is displayed as an EHR plug-in. Operation 408 can include displaying the consolidated view as an EHR plug-in on the plurality of workstations 30. In some examples, operation 408 can include displaying the consolidated view on any one of the display 50 on the patient support apparatus 40, the display 62 of the patient monitoring device 60, and the display 38 mounted on the wall.

The method 400 includes an operation 410 of generating an alert for compliance with the falls risk protocol for the patient P. Examples of the alerts that can be generated in operation 410 are shown in FIGS. 11-21, which are described in more detail below. Operation 410 can include displaying the alert on any one of the plurality of workstations 30, the display 50 on the patient support apparatus 40, the display 62 of the patient monitoring device 60, the display 38 mounted on the wall 14, or on any other display within the healthcare facility 10. A practical real-world advantage of generating the alert for display in operation 410 is that the caregivers are alerted pre-emptively or in real-time of the high fall risk to pre-empt injury when the fall risk of the patient is not known or perceivable to the patient or the caregivers.

FIG. 5 illustrates an example of a consolidated view 500 that can be generated by the data visualization module 108 installed on the risk consolidating system 100. The consolidated view 500 is displayed in accordance with operation 408 of the method 400. The consolidated view 500 displays all data relevant to falls risk on a single screen for a comprehensive falls risk overview. The consolidated view 500 is displayed as a plug-in that is integrated with the EHR 23 of the patient P, which can be displayed on any one of the plurality of workstations 30.

The consolidated view 500 includes patient demographic data 502 that is filtered to display data 503 that is relevant to falls risk such as patient age. The patient demographic data 502 can be acquired from the EHR 23 of the patient P and/or from the HIS 24.

The consolidated view 500 includes patient status data 504 that is filtered to display data 505 that is relevant to falls risk such as medical diagnoses, mental health status, and whether the patient is post-operative. The patient status data 504 can be acquired from the EHR 23 of the patient P and/or from the HIS 24.

The consolidated view 500 includes labs and vitals data 506 that is filtered to display data 507 that is relevant to falls risk such as patient weight, blood pressure, and gait. The labs and vitals data 506 can be acquired from the EHR 23 of the patient P and/or the LIS 26.

The consolidated view 500 includes a list of medications 508 administered to the patient P that is filtered to display data 509 showing medications that can cause dizziness, sedation, confusion, blurred vision, orthostatic hypertension, and/or other side effects that elevate falls risk. The list of medications 508 can be acquired from the EHR 23.

The consolidated view 500 further includes a falls risk summary 510 that includes a falls risk score 512 and factors 514 that contribute to the falls risk score 512 such as mobility, medications, mental status, toileting needs, electrolyte status, and communications/behavior of the patient P. The mobility of the patient P can be measured by the one or more sensors 45 of the patient support apparatus 40, or by the data captured by the camera 70.

As shown in the example provided in FIG. 5, each factor 514 that is used to calculate the falls risk score 512 includes an individual scoring and weighting. For example, the weightings can be shown as bars below the factors 514 with longer bars indicating a heavier weighting than a shorter bars. The bars can also be color coded such as colored green to indicate that a factor 514 has a low scoring such that the factor 514 does not contribute to an elevated falls risk, colored yellow to indicate that a factor 514 has medium score such that the factor 514 moderately contributes to an elevated falls risk, and colored red to indicate that a factor 514 has a high score such that the factor 514 strongly contributes to an elevated falls risk for the patient.

The falls risk summary 510 can further display a status 516 of the patient support apparatus 40 such as whether the siderails 46 are raised in the deployed position or are lowered into the stowed position. In some examples, the status 516 of the patient support apparatus 40 can further include whether it is being occupied by the patient P. In some examples, the falls risk summary 510 can be displayed on the display 50 on the patient support apparatus 40, on the display 62 of the patient monitoring device 60, and/or on the display 38 mounted on the wall 14.

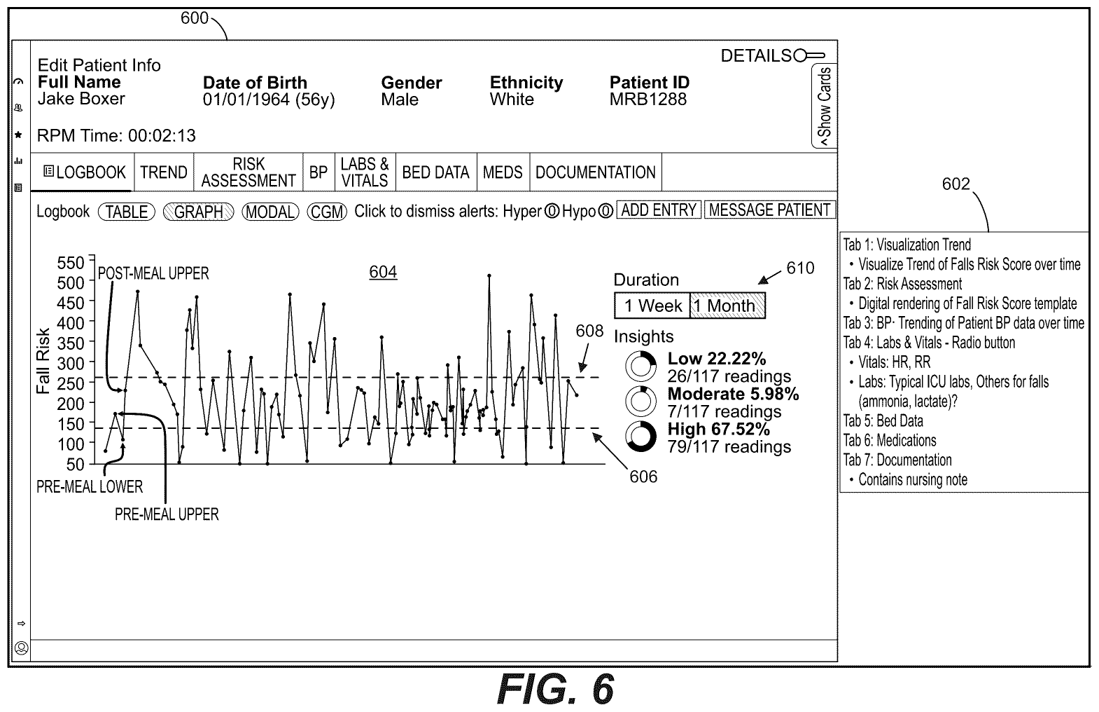

FIG. 6 illustrates another example of a consolidated view 600 that can be generated by the data visualization module 108 installed on the risk consolidating system 100. The consolidated view 600 is displayed in accordance with operation 408 of the method 400. The consolidated view 600 displays all data relevant to falls risk for a comprehensive falls risk overview. The consolidated view 600 is displayed as a plug-in that is integrated with the EHR 23 of the patient P, which can be displayed on any one of the plurality of workstations 30.

The consolidated view 600 includes a plurality of tabs 602 that are each selectable to display a set of data relevant to falls risk. In the example shown in FIG. 6, a first tab is selected for displaying a visualization trend 604 of the falls risk score over time. The visualization trend 604 includes data points when the falls risk score is below a first threshold 606 such that the falls risk score is low, data points when the falls risk score is above the first threshold 606 such that the falls risk score is moderate, and data points when the falls risk score is above a second threshold 608 such that the falls risk score is high. Further, the time period of the visualization trend 604 can be adjusted by selecting a time duration 610 (e.g., 1 week versus 1 month).

Also, the consolidated view 600 displays percentages of when the falls risk score is low, moderate, and high based on the data points included in the visualization trend 604. In the example shown in FIG. 6, the falls risk score is low 22.22% during the selected time period (e.g., 26/117 measurements over 1 month), the falls risk score is moderate 5.98% during the selected time period (e.g., 7/117 measurements over 1 month), and the falls risk is high 67.52% during the selected time period (e.g., 79/117 measurements over 1 month).

As further shown in FIG. 6, the consolidated view 600 can include additional tabs such as a risk assessment tab to display a digital rendering of a falls risk score template that can be used to calculate the falls risk score. The falls risk score template includes a number of data entry fields that receive data for calculating the falls risk score. In some examples, the data entry fields are automatically filled with data relevant to falls risk by the risk consolidating system 100. For example, the mobility of the patient P determined from the one or more sensors 45 on the patient support apparatus 40 and/or from the data captured by the camera 70 can be automatically entered to a data entry field in the falls risk score template for calculating the falls risk score. Alternatively, the data entry fields are manually filled with data relevant to falls risk by caregiver using one of the plurality of workstations 30. In some examples, the falls risk score template is editable by an authorized user such as an administrator in the healthcare facility.

The consolidated view 600 can further include a blood pressure tab that is selectable to display trending of patient blood pressure data over time with threshold alerting for when the blood pressure is lower than a lower limit or when the blood pressure exceeds an upper limit. For example, the trending of patient blood pressure can identify orthostatic hypotension, also called postural hypotension, which is a form of low blood pressure that happens when standing after sitting or lying down. Orthostatic hypotension can cause dizziness or lightheadedness and possibly fainting, and thereby increases the risk of a patient falling.

The consolidated view 600 can further include a labs and vitals tab that is selectable to display vital sign measurements relevant to falls risk such as heart rate and respiration rate. The labs and vitals tab can further display lab results such as for ammonia (NH3) in the blood. High ammonia levels can increase falls risk such as by causing impaired mobility, dizziness, and confusion. Additional lab results can include lactate or lactic acid levels in the blood, which can cause muscle weakness and thereby includes the risk of a patient falling.

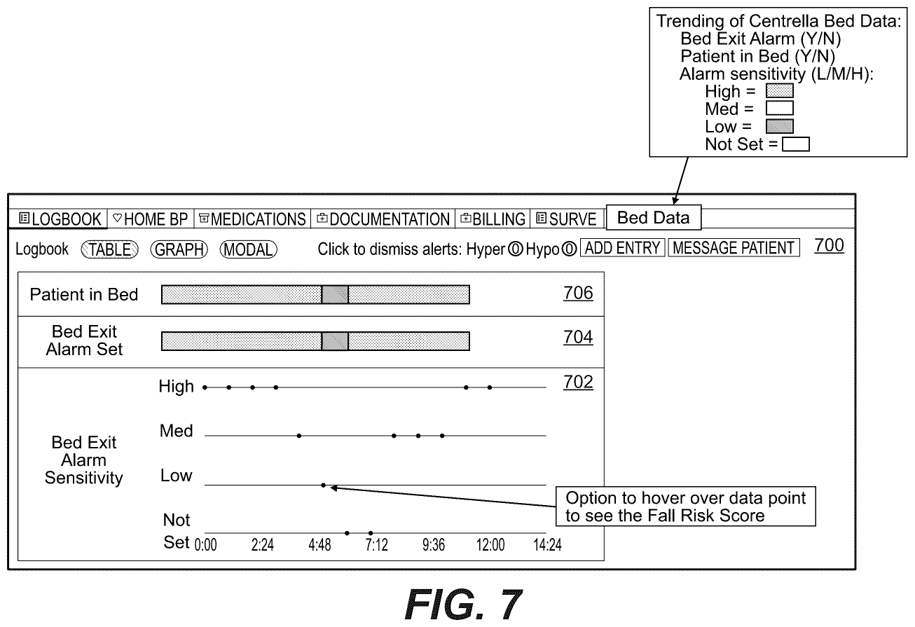

The consolidated view 600 can further include a bed data tab. FIG. 7 illustrates an example of a bed data tab 700. As shown in FIG. 7, the bed data tab 700 when selected displays bed data trended over time such as bed exit alarm sensitivity 702, whether the bed exit alarm is set 704 (yes or no), and whether the patient is in the bed 706 (i.e., the patient support apparatus 40) (yes or no). In some examples, a user can hover a mouse clicker over a data point in the bed exit alarm sensitivity 702 to view a falls risk score calculated at that point in time.

Referring back to FIG. 6, the consolidated view 600 can further include a medications tab that when selected displays medications that are being administered to a patient and that are filtered based on whether the medications affect patient falls risk such as by causing dizziness, sedation, confusion, blurred vision, or orthostatic hypertension.

The consolidated view 600 can further include a documentation tab that includes one or more documentation forms that are customized for assessing or communicating falls risk. The documentation forms can include fall risk care plan for clinical handoff

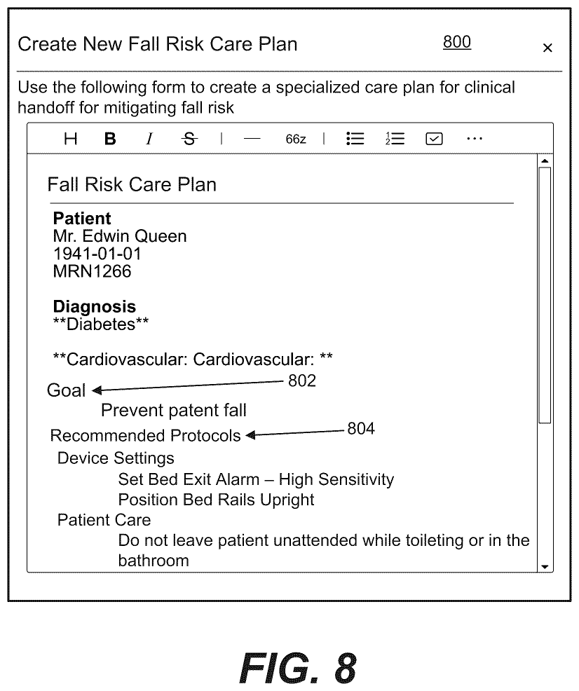

FIG. 8 illustrates an example of fall risk care plan 800 that can be displayed when the documentation tab is selected in the consolidated view 600 of FIG. 6. The fall risk care plan 800 can be used to create a specialized care plan to mitigate falls risk for clinical handoff. The fall risk care plan 800 can identify a patient and one or more diagnoses of the patient. The fall risk care plan 800 further defines a goal 802 for the patient such as prevent patient fall. The fall risk care plan further includes one or more recommended protocols 804 such as device settings (e.g., set bed exit alarm-high sensitivity, position bed rails upright, etc.) and/or patient care instructions (e.g., do not leave patient unattended while toileting or in the bathroom). The goal 802 and the one or more recommended protocols 804 can be edited by a first caregiver when ending his/her shift, and can be viewed by a second caregiver when beginning his/her shift.

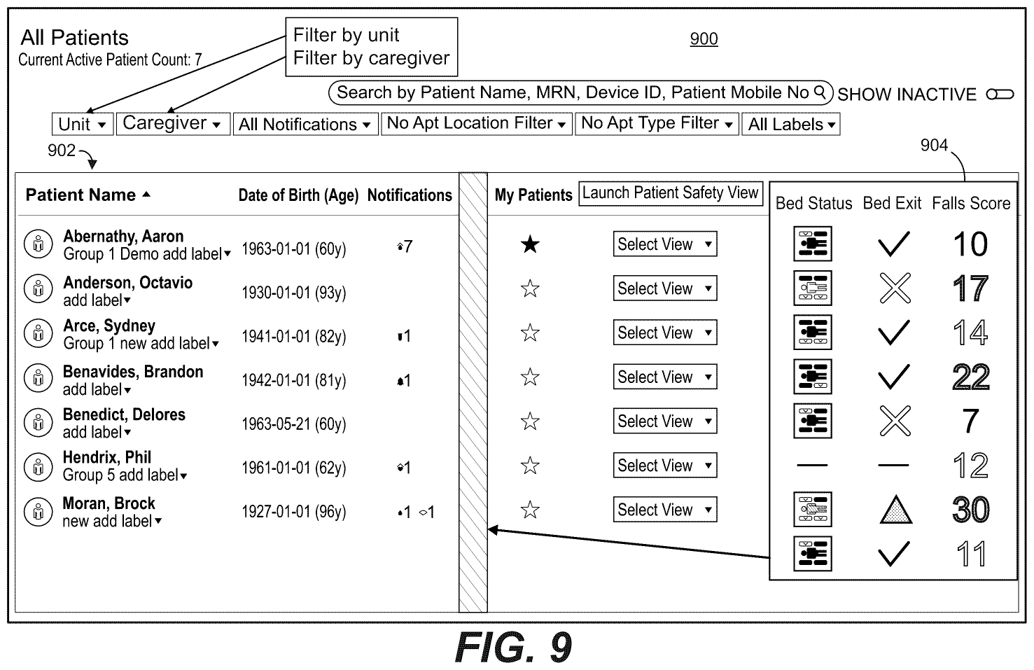

FIG. 9 illustrates an example of a triage view 900 that displays data relevant to falls risk on a single screen for a plurality of patients admitted to the healthcare facility 10. The triage view is generated by the data visualization module 108 installed on the risk consolidating system 100. In some instances, the triage view 900 is displayed as a plug-in that is integrated with the EHR system 22, and which can be displayed on any one of the plurality of workstations 30.

The triage view 900 displays a list of patients 902 that can be filtered by unit within the healthcare facility 10 or by caregiver assignment. For each patient in the list of patients 902, a patient safety view 904 can be selected for displaying a bed status, whether a bed exit alarm is activated or not, and a falls risk score calculated for the patient. The bed status can include a display that shows which siderails 46 are raised to the deployed position to prevent exit from the patient support apparatus 40 by the patient P, and which siderails 46 are lowered into the stowed position to allow exit from the patient support apparatus 40. Further, the bed status can include a display that shows whether or not the patient support apparatus is occupied by a patient.

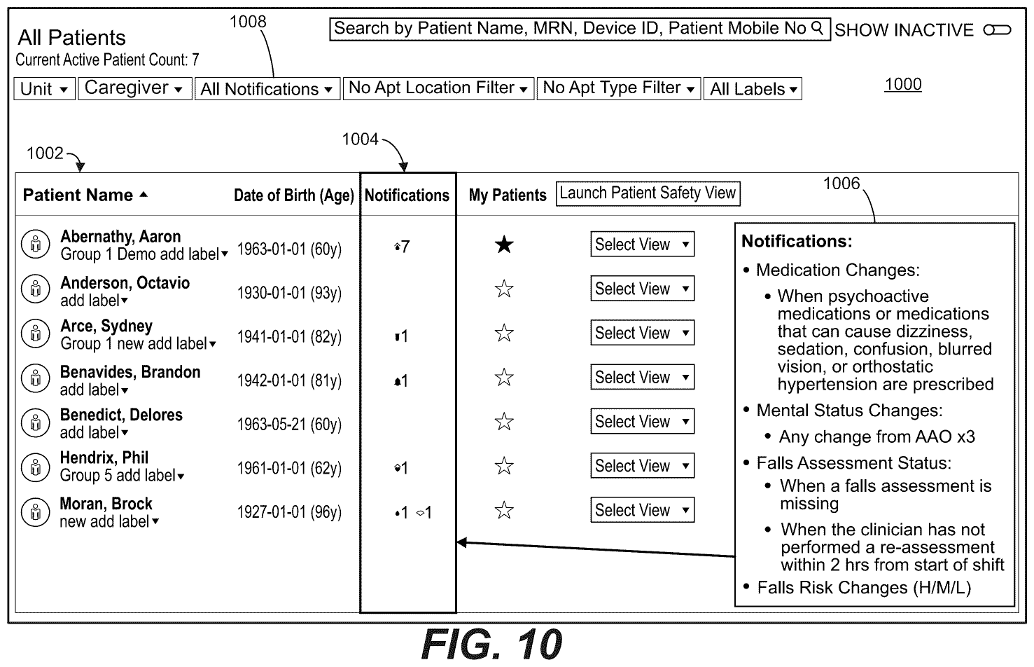

FIG. 10 illustrates another example of a triage view 1000 that can be generated by the data visualization module 108 installed on the risk consolidating system 100. In this example, a notification view 1006 is launched when a notification counter 1004 is selected for a patient included in a list of patients 1002. In the list of patients 1002, the notification counters 1004 can be filtered by one or more selectable criteria 1008 including relevancy to falls risk.

In this illustrative example, the notification view 1006 displays notifications that are filtered based on their relevancy to falls risk such as medication changes (including when psychoactive medications or medications that can cause dizziness, sedation, confusion, blurred vision, or orthostatic hypertension are prescribed); mental status changes (any change away from AAO×3); falls assessment status (when a falls assessment is missing, when a clinician has not performed a re-assessment within 2 hours from start of shift); and falls risk score changes (e.g., changes in falls risk score such as between high, moderation, and low risk).

FIGS. 11-21 illustrate examples of alerts that can be generated by the risk consolidating system 100. The risk consolidating system 100 can utilize artificial intelligence to generate the alerts shown in FIGS. 11-21 based on data acquired from one or more sources such as any one of the healthcare systems 20 and devices inside the patient environment 12.

FIG. 11 illustrates an example of an alert 1100 that can be generated by the risk protocol alert module 110 installed on the risk consolidating system 100. The alert 1100 can be displayed on any one of the plurality of workstations 30. In some examples, the alert 1100 is displayed as an EHR plug-in in the EHR system 22. In some examples, the alert 1100 can be displayed on the display 50 of the patient support apparatus 40, on the display 62 of the patient monitoring device 60, or on the display 38 mounted outside the patient environment 12.

The alert 1100 includes a recommendation 1102 to perform an initial falls risk assessment upon a patient's admission to the healthcare facility 10. The recommendation 1102 is generated from a falls risk protocol that can be retrieved over the network 150 by the risk consolidating system 100 from the protocol database 80. The falls risk protocol can be based on at least one of a geographical location of the healthcare facility 10 (e.g., city or state), a unit within the healthcare facility 10 (e.g., med-surg), and a patient population (e.g., geriatric).

The alert 1100 further includes a first selectable control 1104 to implement the recommendation, and a second selectable control 1106 to setup a reminder for implementing the recommendation. In some examples, selection of the first selectable control 1104 causes the risk consolidating system 100 to automatically implement the recommendation such as by automatically calculating a falls risk score by using health data acquired from a plurality of data sources such as from any of the healthcare systems 20 and/or devices in the patient environment 12 such as the patient support apparatus 40, patient monitoring device 60, and/or camera 70. As described above, the mobility of the patient P can be measured from data collected by the one or more sensors 45 on the patient support apparatus and/or by the data captured by the camera 70, and the mobility of the patient P is used to calculate the falls risk score for the patient P.

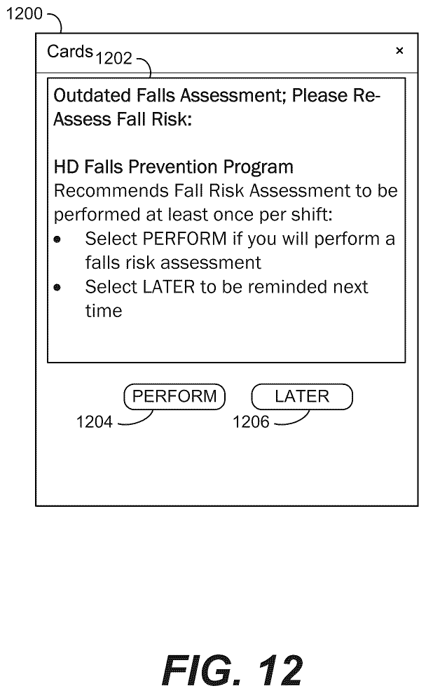

FIG. 12 illustrates another example of an alert 1200 that can be generated by the risk protocol alert module 110 installed on the risk consolidating system 100. The alert 1200 can be displayed on any one of the plurality of workstations 30. In some examples, the alert 1200 is displayed as an EHR plug-in in the EHR system 22. In some examples, the alert 1200 can be displayed on the display 50 of the patient support apparatus 40, on the display 62 of the patient monitoring device 60, or on the display 38 mounted outside the patient environment 12.

The alert 1200 includes a recommendation 1202 that states that a prior falls assessment is outdated. The recommendation 1202 requests reassessment of the falls risk. For example, the recommendation 1202 recommends performing a falls risk upon a start of a new shift by a caregiver such as following patient handoff from another caregiver. The recommendation 1202 is generated from a falls risk protocol that can be retrieved over the network 150 by the risk consolidating system 100 from the protocol database 80.

The alert 1200 includes a first selectable control 1204 to implement the recommendation, and a second selectable control 1206 to setup a reminder for implementing the recommendation. In some examples, selection of the first selectable control 1204 causes the risk consolidating system 100 to automatically implement the recommendation such as by automatically calculating a falls risk score by using health data acquired from a plurality of data sources such as from any of the healthcare systems 20 and/or devices in the patient environment 12 such as the patient support apparatus 40, patient monitoring device 60, and/or camera 70.

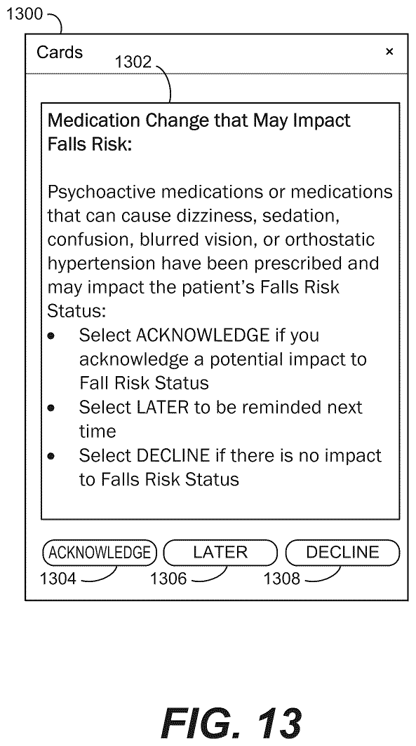

FIG. 13 illustrates another example of an alert 1300 that can be generated by the risk protocol alert module 110 installed on the risk consolidating system 100. The alert 1300 can be displayed on any one of the plurality of workstations 30. In some examples, the alert 1300 is displayed as an EHR plug-in in the EHR system 22. In some examples, the alert 1300 can be displayed on the display 50 of the patient support apparatus 40, on the display 62 of the patient monitoring device 60, or on the display 38 mounted outside the patient environment 12.

The alert 1300 includes a recommendation 1302 for performing a falls risk assessment upon a change in status of a patient such as when a new medication is prescribed to the patient that can cause dizziness, sedation, confusion, blurred vision, or orthostatic hypertension. The recommendation 1302 is generated from a falls risk protocol that can be retrieved over the network 150 by the risk consolidating system 100 from the protocol database 80. The alert 1300 includes a first selectable control 1304 to acknowledge or implement the recommendation, a second selectable control 1306 to setup a reminder to implement the recommendation, and a third selectable control 1308 to decline the recommendation.

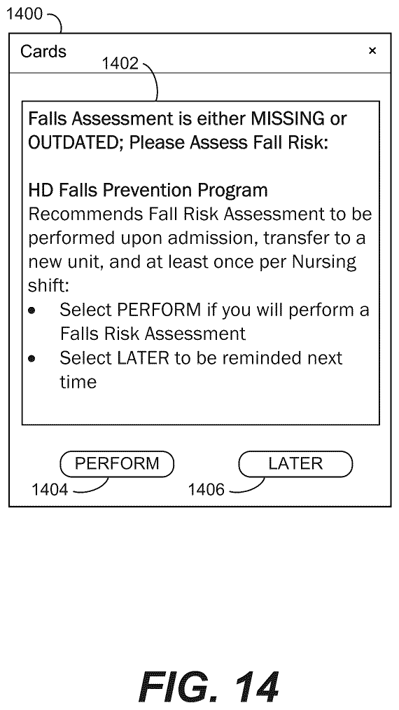

FIG. 14 illustrates another example of an alert 1400 that can be generated by the risk protocol alert module 110 installed on the risk consolidating system 100. The alert 1400 can be displayed on any one of the plurality of workstations 30. In some examples, the alert 1400 is displayed as an EHR plug-in in the EHR system 22. In some examples, the alert 1400 can be displayed on the display 50 of the patient support apparatus 40, on the display 62 of the patient monitoring device 60, or on the display 38 mounted outside the patient environment 12.

The alert 1400 includes a recommendation 1402 to perform a falls risk assessment because a prior falls risk assessment is either outdated or missing. The recommendation 1402 states that falls risk assessment should be performed upon admission to the healthcare facility 10, transfer to another unit within the healthcare facility 10, and at least once per nursing shift. The recommendation 1402 is based on a falls risk protocol retrieved from the protocol database 80.

The alert 1400 includes a first selectable control 1404 to accept the recommendation, and a second selectable control 1406 to setup a reminder regarding implementing the recommendation. In some examples, selection of the first selectable control 1404 causes the risk consolidating system 100 to automatically implement the recommendation such as by automatically calculating a falls risk score by using health data acquired from a plurality of data sources such as from any of the healthcare systems 20 and/or devices in the patient environment 12 such as the patient support apparatus 40, patient monitoring device 60, and/or camera 70.

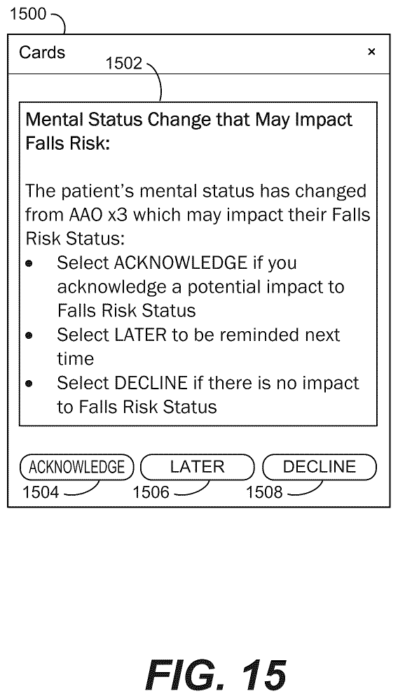

FIG. 15 illustrates another example of an alert 1500 that can be generated by the risk protocol alert module 110 installed on the risk consolidating system 100. The alert 1500 can be displayed on any one of the plurality of workstations 30. In some examples, the alert 1500 is displayed as an EHR plug-in in the EHR system 22. In some examples, the alert 1500 can be displayed on the display 50 of the patient support apparatus 40, on the display 62 of the patient monitoring device 60, or on the display 38 mounted outside the patient environment 12.

The alert 1500 includes a recommendation 1502 to perform a falls risk assessment upon a change in a patient's mental status (e.g., the patient's mental status has changed from AAO (awake, alert, and oriented)×3 which may impact their falls risk score). The recommendation 1502 is based on a falls risk protocol retrieved from the protocol database 80.

The alert 1500 includes a first selectable control 1504 to acknowledge the recommendation, a second selectable control 1506 to setup a reminder to implement the recommendation, and a third selectable control 1508 to decline the recommendation. In some examples, selection of the first selectable control 1504 causes the risk consolidating system 100 to automatically implement the recommendation such as by automatically calculating a falls risk score by using health data acquired from a plurality of data sources such as from any of the healthcare systems 20 and/or devices in the patient environment 12 such as the patient support apparatus 40, the patient monitoring device 60, and/or the camera 70.

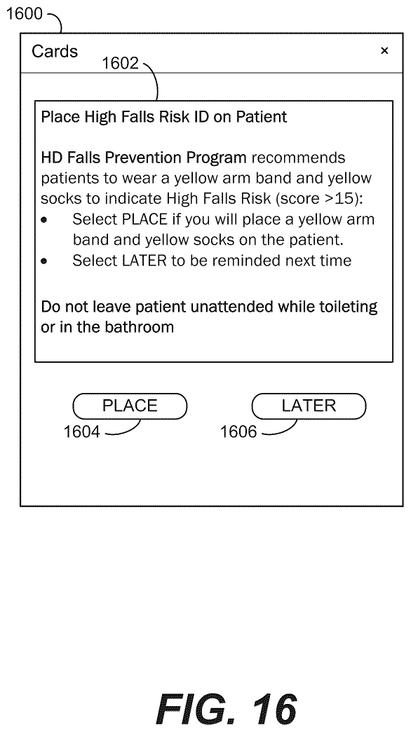

FIG. 16 illustrates another example of an alert 1600 that can be generated by the risk protocol alert module 110 installed on the risk consolidating system 100. The alert 1600 can be displayed on any one of the plurality of workstations 30. In some examples, the alert 1600 is displayed as an EHR plug-in in the EHR system 22. In some examples, the alert 1600 can be displayed on the display 50 of the patient support apparatus 40, on the display 62 of the patient monitoring device 60, or on the display 38 mounted outside the patient environment 12.

The alert 1600 includes a recommendation 1602 to perform an action to mitigate falls risk for a patient having a high falls risk score such as making the patient wear a yellow armband and yellow socks to indicate a high falls risk score, and placing a high falls risk indicator on the display 38 outside the patient environment 12. The recommendation 1602 is based on a falls risk protocol retrieved by the risk protocol alert module 110 from the protocol database 80.

The alert 1600 includes a first selectable control 1604 to accept the recommendation, and a second selectable control 1606 to setup a reminder regarding implementing the recommendation. In some examples, selection of the first selectable control 1604 causes the risk consolidating system 100 to automatically implement the recommendation such as by updating a screen displayed on the display 38 to identify the patient as having a high falls risk.

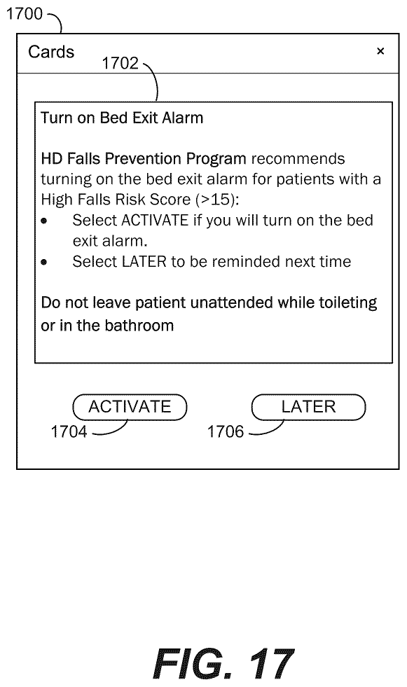

FIG. 17 illustrates another example of an alert 1700 that can be generated by the risk protocol alert module 110 installed on the risk consolidating system 100. The alert 1700 can be displayed on any one of the plurality of workstations 30. In some examples, the alert 1700 is displayed as an EHR plug-in in the EHR system 22. In some examples, the alert 1700 can be displayed on the display 50 of the patient support apparatus 40, on the display 62 of the patient monitoring device 60, or on the display 38 mounted outside the patient environment 12.

The alert 1700 includes a recommendation 1702 to activate the bed exit alarm on the patient support apparatus 40 to mitigate falls risk. The recommendation 1702 is based on a falls risk protocol retrieved by the risk protocol alert module 110 from the protocol database 80.

The alert 1700 includes a first selectable control 1704 to implement the recommendation, and a second selectable control 1706 to setup a reminder to implement the recommendation. In some examples, selection of the first selectable control 1704 causes the risk consolidating system 100 to automatically perform the recommendation such as by activating the bed exit alarm on the patient support apparatus 40 by communication over the network 150.

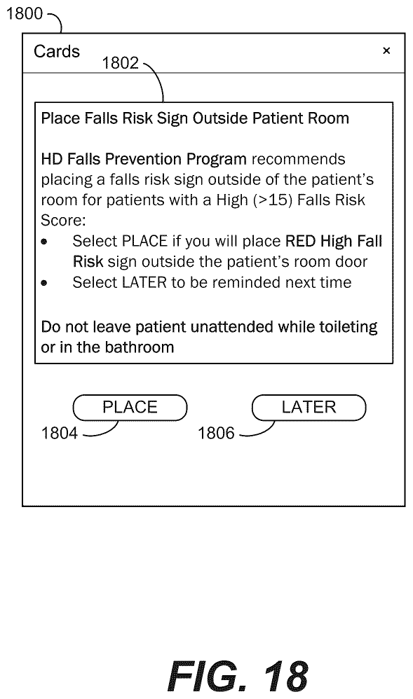

FIG. 18 illustrates another example of an alert 1800 that can be generated by the risk protocol alert module 110 installed on the risk consolidating system 100. The alert 1800 can be displayed on any one of the plurality of workstations 30. In some examples, the alert 1800 is displayed as an EHR plug-in in the EHR system 22. In some examples, the alert 1800 can be displayed on the display 50 of the patient support apparatus 40, on the display 62 of the patient monitoring device 60, or on the display 38 mounted outside the patient environment 12.

The alert 1800 includes a recommendation 1802 for adjusting the display 38 outside of the patient environment 12 to indicate that the patient has a high falls risk such as by displaying the color red on a screen displayed on the display 38. The recommendation 1802 is based on a falls risk protocol retrieved from the protocol database 80. Displaying data in this way immediately assists caregivers to allocate resources to high fall risk patients to pre-empt injury.

The alert 1800 includes a first selectable control 1804 to implement the recommendation, and a second selectable control 1806 to setup a reminder to implement the recommendation. In some examples, selection of the first selectable control 1804 causes the risk consolidating system 100 to automatically perform the recommendation such as by changing the color of the screen displayed on the display 38 to red, or to display some other type of symbol or indicator that the patient admitted to the patient environment 12 has a high falls risk.

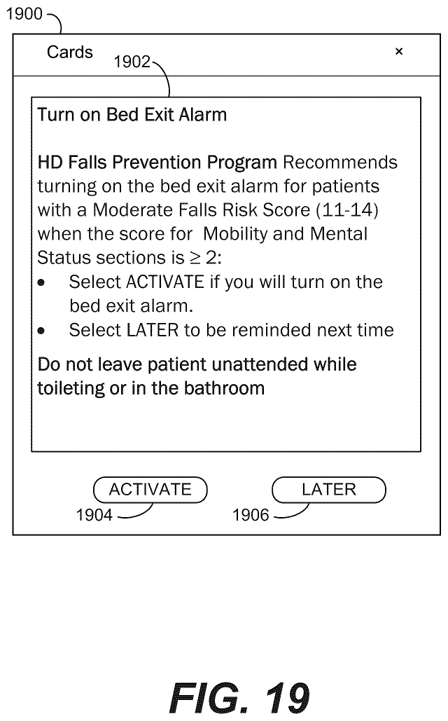

FIG. 19 illustrates another example of an alert 1900 that can be generated by the risk protocol alert module 110 installed on the risk consolidating system 100. The alert 1900 can be displayed on any one of the plurality of workstations 30. In some examples, the alert 1900 is displayed as an EHR plug-in in the EHR system 22. In some examples, the alert 1900 can be displayed on the display 50 of the patient support apparatus 40, on the display 62 of the patient monitoring device 60, or on the display 38 mounted outside the patient environment 12.

The alert 1900 includes a recommendation 1902 to perform an action to mitigate falls risk for a patient having a moderate falls risk score and a score for mobility and mental status is above a predetermined threshold. In this example, the action includes activating the bed exit alarm on the patient support apparatus 40. The recommendation 1902 is based on a falls risk protocol retrieved from the protocol database 80. The alert 1900 includes a first selectable control 1904 to implement the recommendation, and a second selectable control 1906 to setup a reminder to implement the recommendation. In some examples, selection of the first selectable control 1904 causes the risk consolidating system 100 to automatically perform the recommendation such as by activating the bed exit alarm on the patient support apparatus 40 by communicating with the patient support apparatus 40 over the network 150.

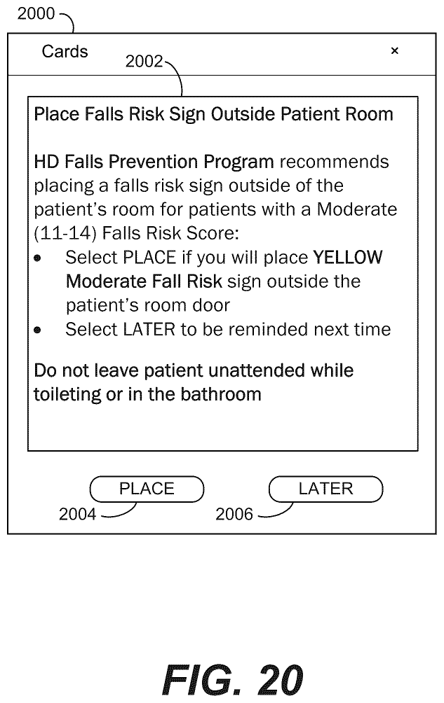

FIG. 20 illustrates another example of an alert 2000 that can be generated by the risk protocol alert module 110 installed on the risk consolidating system 100. The alert 2000 can be displayed on any one of the plurality of workstations 30. In some examples, the alert 2000 is displayed as an EHR plug-in in the EHR system 22. In some examples, the alert 2000 can be displayed on the display 50 of the patient support apparatus 40, on the display 62 of the patient monitoring device 60, or on the display 38 mounted outside the patient environment 12.

The alert 2000 includes a recommendation 2002 to perform an action to mitigate falls risk for a patient having a moderate falls risk score. In this example, the action includes altering the display 38 mounted on the wall 14 outside of the patient environment 12 to display an indicator of the moderate falls risk score such as by displaying the color yellow the display 38. The recommendation 2002 is generated from a falls risk protocol that can be retrieved over the network 150 by the risk consolidating system 100 from the protocol database 80.

The alert 2000 includes a first selectable control 2004 to implement the recommendation, and a second selectable control 2006 to setup a reminder to implement the recommendation. The selection of the first selectable control 2004 can cause the risk consolidating system 100 to automatically perform the recommendation by changing the color of the screen displayed on the display 38 to yellow, or to display some other type of symbol or indicator that the patient admitted to the patient environment 12 has a moderate falls risk.

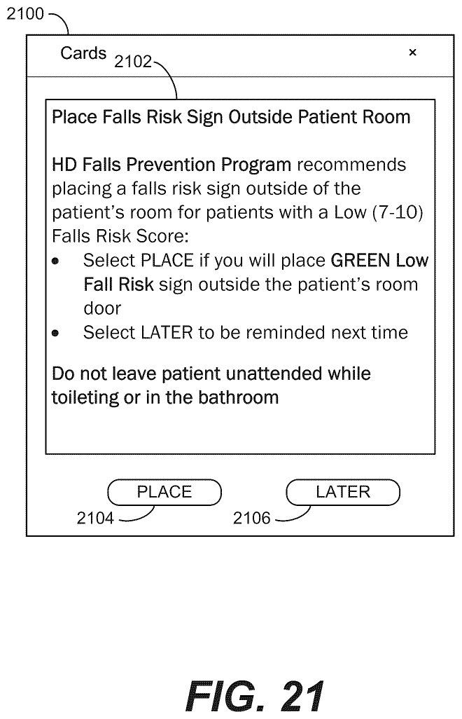

FIG. 21 illustrates another example of an alert 2100 that can be generated by the risk protocol alert module 110 installed on the risk consolidating system 100. The alert 2100 can be displayed on any one of the plurality of workstations 30. In some examples, the alert 2100 is displayed as an EHR plug-in in the EHR system 22. In some examples, the alert 2100 can be displayed on the display 50 of the patient support apparatus 40, on the display 62 of the patient monitoring device 60, or on the display 38 mounted outside the patient environment 12.

The alert 2100 includes a recommendation 2102 for performing an action to mitigate falls risk for a patient having a low falls risk score. In this example, the action includes altering the display 38 mounted on the wall 14 outside of the patient environment 12 to display an indicator of the low falls risk score such as by displaying the color green the display 38. The recommendation 2102 is generated from a falls risk protocol that can be retrieved over the network 150 by the risk consolidating system 100 from the protocol database 80.

The alert 2100 includes a first selectable control 2104 to implement the recommendation, and a second selectable control 2106 to setup a reminder to implement the recommendation. The selection of the first selectable control 2104 can cause the risk consolidating system 100 to automatically perform the recommendation by changing the color of the screen displayed on the display 38 to green, or to display some other type of symbol or indicator that the patient admitted to the patient environment 12 has a low falls risk.

The various embodiments described above are provided by way of illustration only and should not be construed to be limiting in any way. Various modifications can be made to the embodiments described above without departing from the true spirit and scope of the disclosure.

What is claimed is:

1. A system for improving falls risk protocol compliance, the system comprising:
at least one processing device; and
at least one computer readable data storage device storing software instructions that, when executed by the at least one processing device, cause the at least one processing device to:
receive health data from a plurality of data sources;
identify data relevant to a falls risk for a patient;
identify a falls risk protocol for the patient;
display the data relevant to the falls risk for the patient in a consolidated view; and
generate an alert for compliance with the falls risk protocol for the patient.

2. The system of claim 1, wherein the plurality of data sources include data stored in one or more healthcare systems including at least one of an electronic health record system, a hospital information system, a laboratory information system, and a nurse call system.

3. The system of claim 1, wherein the consolidated view is displayed within an electronic health record associated with the patient.

4. The system of claim 1, wherein the plurality of data sources include one or more devices positioned inside a patient environment, the one or more devices including at least one of a patient support apparatus, a camera, and a patient monitoring device.

5. The system of claim 4, wherein the alert is displayed on at least one of a display of the patient support apparatus, a display of the patient monitoring device, and a display mounted on a wall proximate to an entrance of the patient environment.

6. The system of claim 1, wherein the instructions, when executed by the at least one processing device, further cause the at least one processing device to:
display a triage view having a list of patients and a notification counter for each patient in the listing of patients, the notification counter filtered based on relevancy to the falls risk.

7. The system of claim 6, wherein the notification counter is automatically updated based on changes to medication, mental status, falls assessment status, and falls risk.

8. The system of claim 1, wherein the falls risk protocol for the patient is identified based on at least one of a geographical location of a healthcare facility, a unit within the healthcare facility, and a patient population.

9. The system of claim 1, wherein the instructions, when executed by the at least one processing device, further cause the at least one processing device to:
display a template for creating a fall risk care plan for patient handoff.

10. The system of claim 1, wherein the alert includes a recommendation to perform a falls risk assessment, a recommendation to update the falls risk assessment, or a recommendation to perform an action to mitigate falls risk for the patient.

11. The system of claim 10, wherein the alert includes one or more selectable controls to acknowledge, accept, decline, or setup a reminder regarding the implementation of the recommendation.

12. A computer-implemented method for improving falls risk protocol compliance, the method comprising:
receiving health data from a plurality of data sources;
identifying data relevant to a falls risk for a patient;
identifying a falls risk protocol for the patient;
displaying the data relevant to the falls risk for the patient in a consolidated view; and
generating an alert for compliance with the falls risk protocol for the patient.

13. The computer-implemented method of claim 12, wherein the plurality of data sources include data stored in one or more healthcare systems including at least one of an electronic health record system, a hospital information system, a laboratory information system, and a nurse call system.

14. The computer-implemented method of claim 12, further comprising:
displaying the consolidated view in an electronic health record of the patient.

15. The computer-implemented method of claim 12, further comprising:
displaying the alert on at least one of a display of a patient support apparatus, a display of a patient monitoring device, and a display mounted on a wall.

16. The computer-implemented method of claim 12, further comprising:
displaying a triage view having a list of patients and a notification counter for each patient in the listing of patients; and
filtering the notification counters based on relevancy to the falls risk.

17. The computer-implemented method of claim 12, further comprising:
identifying the falls risk protocol based on at least one of a geographical location of a healthcare facility, a unit within the healthcare facility, and a patient population.

18. The computer-implemented method of claim 12, further comprising:
displaying a template for creating a fall risk care plan for patient handoff.

19. The computer-implemented method of claim 12, wherein the alert includes a recommendation to perform a falls risk assessment, a recommendation to update the falls risk assessment, or a recommendation to perform an action to mitigate falls risk for the patient.

20. The computer-implemented method of claim 19, further comprising:
displaying the alert to include one or more selectable controls to acknowledge, accept, decline, or setup a reminder regarding the implementation of the recommendation.
